(19) United States
(12) Patent Application Publication
(10) Pub. No.: US 2024/0263118 A1
(43) Pub. Date: Aug. 8, 2024

(54) A SYSTEM FOR CELL CULTURING

(71) Applicant: TAMPERE UNIVERSITY FOUNDATION SR, Tampereen Yliopisto (FI)

(72) Inventors: Arri PRIIMÄGI, Tampereen Yliopisto (FI); Chiara FEDELE, Tampereen Yliopisto (FI); Teemu IHALAINEN, Tampereen Yliopisto (FI)

(21) Appl. No.: 18/692,161

(22) PCT Filed: Aug. 24, 2022
(86) PCT No.: PCT/FI2022/050549

§ 371 Date: Mar. 14, 2024

(30) Foreign Application Priority Data
Oct. 13, 2021 (FI) 20217156

(51) Int. Cl. C12M 1/00; C12M 1/12

(52) U.S. Cl. CPC C12M 23/20; C12M 25/06; C12M 41/06

(57) ABSTRACT

The present disclosure relates systems for cell culturing, in particular to light-reconfigurable systems including a support substrate, an azobenzene-containing layer and a protective coating layer. The disclosure relates also reversibly inscribing topographies on surface of the azobenzene-containing material of the system, a method for culturing cells on the system and to cell cultures obtainable by the method. Also, a method for producing the system is disclosed.

### FIELD

The present disclosure relates systems for cell culturing, in particular to light-reconfigurable systems comprising bilayer structures comprising an azobenzene-containing layer and a protective coating layer.

### BACKGROUND

In drug development, over 90% of the new molecules developed fail during the clinical phase tests. The major cause for the low success rate is the cellular models used in in vitro studies. These model systems are not able to reproduce the natural conditions of the human body: in vivo, the cellular microenvironment regulates various cellular functions. Thus, there is a clear need for better cell-based models—to do this, it is important to mimic the dynamic in vivo conditions in the in vitro cell culture as closely as possible, including the cellular microenvironment.

Currently, cells are cultured mostly in cell culture discs, flasks and plates without proper simulation of the dynamic in vivo microenvironment of the cells, the cellular niche. One major drawback in these conditions is the lack of surface features, i.e., surface topography, which does not reflect well the environment that cell encounters in human body. In addition, adherent cells in our bodies are continuously modifying and interacting with their surroundings. This behavior creates dynamic extracellular environment, which is difficult to recapitulate in vitro.

Different microengineering approaches have been used to construct micropatterned cell culture substrates, but they usually feature square topographies instead of smooth structures, which does not resemble the cellular environment. Furthermore, they still suffer from lack of reconfigurability or dynamic changes of the surface and thus offer only static environment for the cells.

Accordingly, there is still need for reconfigurable surfaces for cell cultures.

### SUMMARY

The present invention is based on the observation that surface topography can be repeatedly altered, erased or generally reconfigured by creating light-induced surface features using bilayer structures comprising an azobenzene-containing layer and a protective coating layer.

Accordingly, it is an object of the present invention to provide a patterned system for cell culturing according to claim 1.

It is also an object of the present invention to provide a method for reversibly inscribing topographies on surface of an azobenzene-containing material of the system, the method comprising focusing beam of light to the material or projecting an interference pattern of laser light to the material.

It is still an object of the present invention to provide a method for erasing topographical features of the patterned system, the method comprising subjecting the topographic features to light of 430 nm-600 nm generated by a laser, preferably continuous wave laser, or generated by a fluorescent lamp or generated by a LED.

It is still an object of the present invention to provide use of the system or the patterned system as a cell culture platform.

It is still an object of the present invention to provide a method for cell culturing, wherein the cells are cultured on the patterned system.

Further objects of the present invention are described in the accompanying dependent claims.

Exemplifying and non-limiting embodiments of the invention, both as to constructions and to methods of operation, together with additional objects and advantages thereof, are best understood from the following description of specific exemplifying embodiments when read in connection with the accompanying drawings.

The verbs “to comprise” and “to include” are used in this document as open limitations that neither exclude nor require the existence of un-recited features. The features recited in the accompanied depending claims are mutually freely combinable unless otherwise explicitly stated. Furthermore, it is to be understood that the use of “a” or “an”, i.e., a singular form, throughout this document does not exclude a plurality.

### BRIEF DESCRIPTION OF DRAWINGS

FIG. 1 shows a system according to an exemplary non-limiting embodiment of the present invention.

FIG. 2 demonstrates the trans-cis isomerization reaction of an azobenzene derivative induced by light.

FIG. 3 shows graphical presentation of azobenzene-driven a) surface relief grafting formation and b) erasure of an exemplary system of the present invention.

FIG. 4 demonstrates sinusoidal surface relief gratings inscribed by interference lithography, atomic force microscope 3D projection, average cross-sectional profile and topography inscribed by laser scanning of an exemplary system of the present invention.

FIG. 5 demonstrates morphological difference between Madin Darby Canine Kidney epithelial cells on flat (left) and on 1 μm period sinusoidal topography (right) of an exemplary system of the present invention comprising azobenzene-based films.

FIG. 6a shows diffraction efficiency (DE) curves (averaged over three measurements for each curve) with different thicknesses of PDMS, during surface relief gratings (SRG) inscription with intensity of 300 mW cm⁻² (488 nm, circular polarization, probe beam wavelength 633 nm). Standard deviations for the DE values at the end of the SRG inscription are ±7% (DR1g), 0.5% (DR1g-PDMS₅₀), +5% (DR1g-PDMS₁) and ±6% (DR1g-PDMS_{0.02})

FIG. 6b shows AFM images of the surface topography on DR1g-1 PDMS_{0.02} after SRG inscription.

FIG. 6c shows AFM images of the surface topography on DR1g-PDMS₁ after SRG inscription.

FIG. 7 shows diffraction efficiency curves for different thicknesses of PDMS layer during a) SRG inscription with intensity of 500 mW cm⁻² and b) SRG erasure with a 530 nm LED; c) AFM images of the surface topography of DR1g-PDMS₁ after inscription (top) and erasure (bottom); d) Cross-sectional profiles of the SRG modulation depth for photo inscribed (solid line) and erased (dashed line) DR1g-PDMS₁.

FIG. 8 shows resistance of a non-coated DR1g (left) and a DR1g coated with a 90 nm layer of parylene C (right) to acetone.

FIG. 9 shows a) graphical representation of the polyacrylamide hydrogel coating on surface modulated DR1g; and b) DIC analysis via the fluorescent microparticles of the traverse strain induced in the hydrogel by DR1g photo stimulation.

FIG. 10 shows a) schematic representation of DR1g-PDMS₁ sample preparation for cell culture experiments; b) optical microscopy images of MDCK II cells on a flat glass substrate and surface-patterned films of DR1g and DR1g-PDMS₁ after 24 h from cell seeding. Black arrow indicates the SRG topography direction. Scale bars: 50 μm; c) Immunolabeled MDCK II cells on surface-patterned DR1g-PDMS₁ bilayer at different time points (24 h, 72 h). The labels used were DAPI (chromatin), E-cadherin (cell-cell junctions) and pFAK (mature focal adhesions). The organization of focal adhesions was analyzed with fast Fourier transform (FFT) of the pFAK image showing the periodicity in the image (indicated by 1st order peaks). Black arrow indicates the SRG topography direction. Scale bars 20 μm.

FIG. 11 shows DHM images of SRG topography on DR1g-PDMS₁ after erasure with a fluorescent lamp of a confocal microscope filtered in the blue region (470±40 nm) a) in dry environment and b) in liquid environment. Irradiation time: 5 min. Scale bars: 10 μm. Surface profiles of the SRG topography on DR1g-PDMS₁ after erasure c) in dry environment and d) in liquid environment.

### DESCRIPTION

According to one aspect the present disclosure concerns a system for cell culturing. An exemplary system 100 is shown in FIG. 1. The system comprises a support structure 101, an azobenzene-containing middle layer 102, and a top layer 103.

The support structure can be any support structure used for cell culturing. Exemplary support structures are cell culture disc such as petri dish, microscope coverslip and a well plate. The support structure is typically made of plastic or glass. Exemplary petri dish formats are poly(styrene) and glass-bottom petri dish.

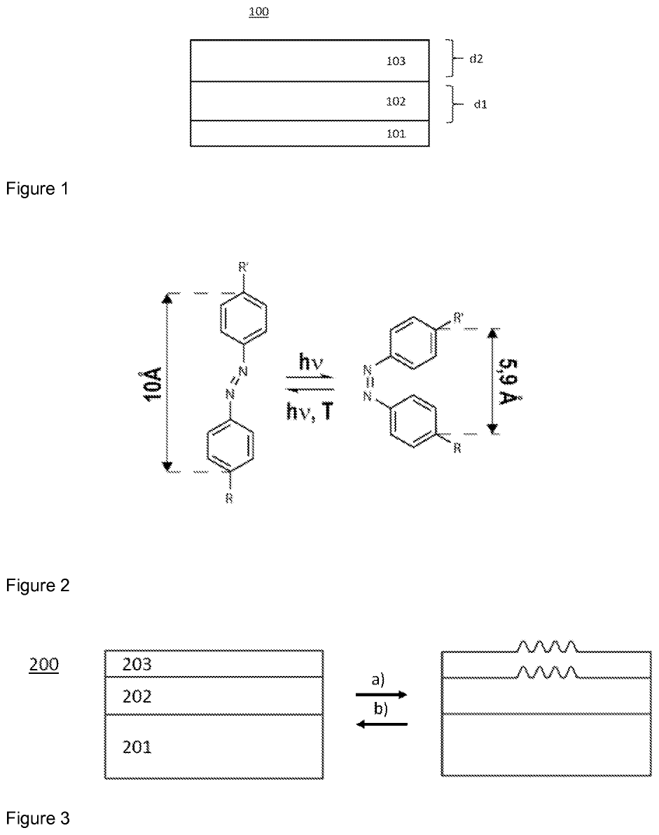

Azobenzene-containing materials are light-reconfigurable. As defined herein a light-reconfigurable material is a material whose shape is reconfigurable upon exposure to light. The azobenzene molecules may be substituted or unsubstituted. Light-induced transformation of an exemplary azobenzene unit is shown in FIG. 2 where R and R′ refer to different para-substituents. Different substituents can also be added to the meta- and ortho-positions. An exemplary azobenzene suitable for the present technology is ethyl-N-(2-hydroxyethyl)-4-(4-nitrophenylazo)aniline.

The top layer comprises protective polymer such as elastomer or hydrogel. Exemplary elastomers are siloxane-containing polymers such as polydimethylsiloxane, PDMS.

According to another embodiment the protective polymer comprises parylenes, preferably parylene C, i.e., poly(chloro-para-xylylene). A variety of substituted [2.2]para-cyclophanes exist, whereby functional groups may be introduced into the phenyl rings. These functional groups allow for the deposition of functionalized parylene films, or they can undergo further functionalization allowing immobilization of bioactive molecules.

PDMS and parylene C are preferable coatings due to their good mechanical and barrier properties, hydrophobicity, chemical resistance, and biocompatibility. Further advantage of parylene C is that it can be deposited to produce pinhole-free ultra-thin films. Furthermore, despite being semicrystalline, parylene C is highly transparent in the thickness of range of interest.

According to one embodiment thickness d₁ of the azobenzene-containing layer is from 50 nm to 5 μm, and thickness d₂ of the top layer is from 20 nm to 100 μm, preferably from 20 nm to 200 nm.

According to another embodiment thickness d₁ of the azobenzene-containing layer is from 50 nm to 5 μm, and thickness d₂ of the top layer is from 50 nm to 100 μm. An exemplary thickness of the azobenzene-containing layer is 500 nm.

When the protective polymer is PDMS or parylene C, thickness of the top layer is preferably below 90 nm.

When the protective polymer is hydrogel, thickness of the top layer is preferably below 50 μm.

The present disclosure also concerns a method to produce a system 100 for cell culturing, the method comprising
a) providing a support structure 101,
b) coating the support structure with an azobenzene-containing layer 102, and
c) coating the azobenzene-containing layer with a top layer 103 comprising a protective polymer.

The support structure is preferably selected from a petri dish, microscopy coverslip, well plate. According to one embodiment the protective polymer comprises hydrogel or an elastomer. Exemplary elastomers are siloxanes. A particular siloxane is PDMS.

According to another embodiment the protective polymer comprises parylene. A particular parylene is parylene C.

According to one embodiment the coating of step b) comprises spin coating.

According to another embodiment the coating of step c) comprises spin coating. Spin coating is preferable when the protective polymer is siloxane such as PDMS.

According to another embodiment the coating of step c) comprises chemical vapor deposition polymerization. This is preferable method when the protective polymer comprises parylene, such as parylene C.

Micro- and sub-micrometer scale topographies can be reversibly inscribed on the surface of the azobenzene-containing material, e.g., by means of light interference lithography, digital micromirror devices, micro lens arrays or simply by scanning a laser beam (for instance, from a laser scanning microscope) over the film surface. In presence of a focused beam of light, azobenzene-based thin coatings tend to accumulate within or escape from the focal volume of the light beam. Therefore, the scanning motion of the light beam allows for the inscription of any shape, just like a drawing tool.

Schematic presentation of producing topographical features to and erasing from an exemplary system of the present invention is shown in FIG. 3.

Accordingly, it is also an aspect the present disclosure to provide method for reversibly inscribing topographies on surface of the azobenzene-containing material of the system. According to one embodiment the method comprises focusing beam of light to said material. According to a particular embodiment the method comprises scanning laser beam over the azobenzene-containing layer.

According to another embodiment the method for reversibly inscribing topographies on surface of the azobenzene-containing material of the system utilizes interference lithography. According to this embodiment the method comprises projecting an interference pattern of laser light to the material.

Since the middle layer is coated with the top layer, the topographies are formed on the top layer also.

The reversibly inscribing comprises patterning and erasing. Wavelength of the light used for patterning and erasing is typically 400 nm-600 nm, preferably 430 nm-530 nm. The appropriate intensity range depends on the technique used. For example, 100 mW cm^{-2 600} mW cm⁻² is sufficient for interference lithography, whereas about 1 W cm⁻²-5 W cm⁻² are used for the patterning/erasure at the laser scanning confocal microscope.

According to one embodiment, for patterning the method comprises subjecting one or more areas of the azobenzene-containing layer to 400 nm-600 nm, preferably 430 nm-530 nm light generated by a laser, preferably a continuous wave laser thereby producing topographical features to the system. Exemplary intensity of the light generated by the laser is preferably from 1 W cm⁻² to 5 W cm⁻².

According to embodiment, for erasing, the topographical features of the patterned system are subjected to 400 nm-600 nm, preferably 430 nm-530 nm light generated by a laser, preferably a continuous wave laser. Exemplary intensity of the light generated by a laser is from 1 W cm⁻² to 5 W cm⁻².

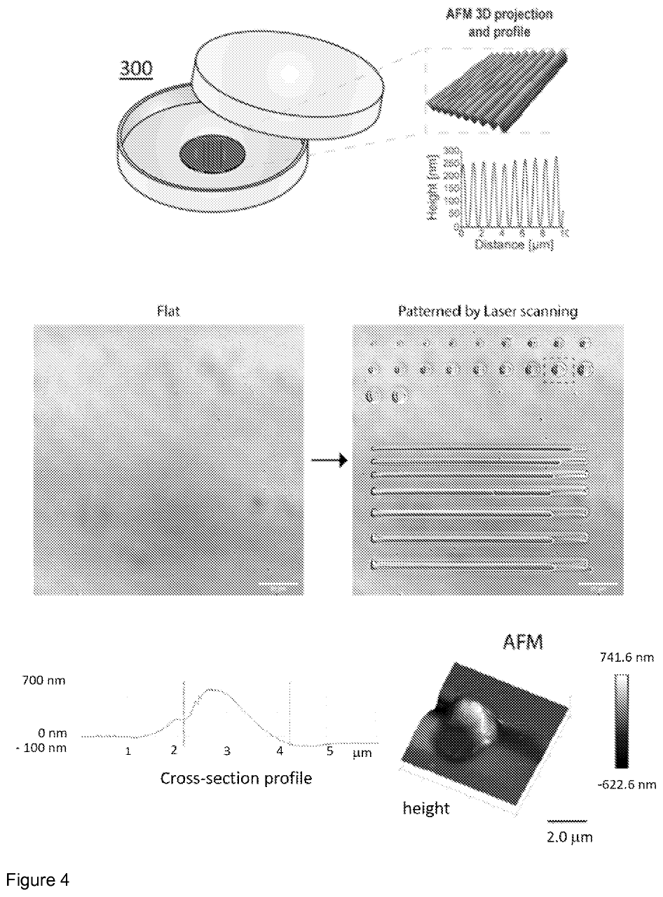

An exemplary system 300 of the present disclosure obtainable by the method disclosed above is shown in FIG. 4. The figure demonstrates also sinusoidal surface relief gratings inscribed by interference lithography, atomic force microscope 3D projection, average cross-sectional profile and topography inscribed by laser scanning of the system.

According to another aspect the present disclosure concerns a method for patterning the system by subjecting one or more areas of the azobenzene-containing layer of system, to 400 nm-600 nm, preferably 430 nm-530 nm light generated by a laser, preferably a continuous wave laser, thereby producing topographical features to the system. Intensity of the light is typically from 1 W cm⁻² to 5 W cm⁻². Exemplary wavelength is 488 nm which is preferable wavelength when the azobenzene-containing material is ethyl-N-(2-hydroxyethyl)-4-(4-nitrophenylazo)-aniline. Exemplary intensity is 1 W cm⁻².

The present disclosure also concerns a method for erasing the topographical features from the system by subjecting the topographic features to 460 nm-530 nm light generated by a laser, preferably a continuous wave laser. Exemplary wavelengths are 470 nm, 488 nm, and 530 nm. According to another embodiment the erasing is performed by using light generated by a fluorescent lamp filtered in the range 430 nm-530 nm. Exemplary intensity generated by the laser is 1 W cm⁻². Intensity of the light generated by the fluorescent lamp is typically from 1 W cm⁻² to 5 W cm⁻². The erasing can be performed using the above-mentioned laser and fluorescent lamp and also by LED.

The invention allows free-form topographical patterns to be created on a cell culture substrate. The topography can further be erased with a uniform light source (e.g., fluorescent lamp, LED light or a laser) even when the cells are already growing on the substrate and re-written to create a new pattern to the culture dish, making it possible to create dynamic topographies, better mimicking the dynamic conditions in human body.

Thus, it is still an aspect of the present disclosure to provide a method for culturing cells on the system comprising topographic features on top surface. The system comprising topographic features is obtainable as disclosed above.

According to an exemplary embodiment, the method comprises the following steps
a) providing the patterned system,
b) coating top layer of the patterned system with cell adhesive proteins, and
c) seeding the cells on the cell-adhesive proteins.

According to a preferable embodiment the method comprises subjecting the system to oxygen plasma treatment prior to step b).

Exemplary cells are selected from a group consisting of epithelial cells, fibroblasts, endothelial cells, neurons, mesenchymal stem cells, astrocytes, cardiomyocytes, and cancer cells.

Exemplary cell adhesive proteins suitable for the method are selected from a group consisting of collagen, fibronectin, and laminin. The selection of the cell adhesive protein is dependent on the cell type to be seeded.

The system of the present invention allows robust reconfigurable control of surface topography, based on creating light-induced surface features using azobenzene-containing bilayers. According to one embodiment a cell culture disc is coated with azobenzene-containing polymer film presenting a protective siloxane layer allowing easy chemical modification of the surface, ensuring biocompatibility, and fully supporting existing protein deposition techniques. According to another embodiment a cell culture disc is coated with azobenzene-containing polymer film presenting a protective parylene C layer allowing easy chemical modification of the surface, ensuring biocompatibility, and fully supporting existing protein deposition techniques.

In vivo, cellular dynamic interplay with the surrounding extracellular matrix (ECM) plays a key role in the regulation of many physiological and pathological processes, such as tissue morphogenesis, healing, and tumor growth. The present invention allows for real-time control of the extracellular niche, (multi)cellular spatial arrangement, orientation, and migration, due to the possibility to manipulate the surface features with light. The process is reversible, remotely controllable, and non-invasive, which would be important for definition of chrono-programs for e.g., cell differentiation, stem cell phenotype harvesting, tissue regeneration and triggering cell directional migration as well as decoupling topographic and chemical cues. In the body, cells are exposed to different kinds of biophysical cues that can be converted into biochemical activity in a process called mechanotransduction. These cues are important co-regulators of e.g., cell alignment and migration. For example, muscle, neuronal, and endothelial cells show highly aligned organization in our bodies and cell migration is greatly influenced by the topographical features of the cellular environment.

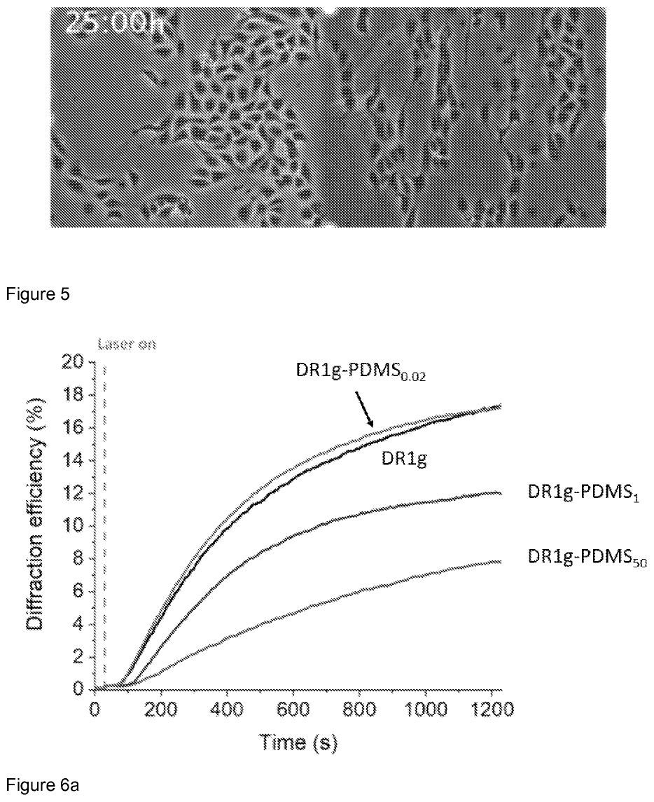

To demonstrate the feasibility of the light-induced patterning and its effect on cells, epithelial cells were cultured on a flat surface (FIG. 5, left) and surface of a system comprising light-induced azobenzene-containing material (FIG. 5, right). As seen from the figure, the surface topography has a significant effect on the cell alignment and migration.

### Results and Discussion

Characterization of SRG Inscription and Erasure on DR1g-PDMS Bilayer

### Structures

To assemble the platform, glass coverslips were first coated with photopatternable amorphous thin layer of ethyl-N-(2-hydroxyethyl)-4-(4-nitrophenylazo)-aniline i.e., Disperse Red 1-containing molecular glass (DR1g; thickness 480±20 nm) that were further coated with PDMS. The resulting DR1g-PDMS bilayer structure was used as the light-responsive cell culturing platform where DR1g functions as the light-responsive part.

To study the effect of the PDMS (base: curing agent ratio 10:1) on the SRGs formation, three different PDMS pre-polymer dilutions in n-hexane (0.02, 1, and 50 wt-%) were tested with the same spin coating parameters. These samples are denoted here as DR1g-PDMSx, where x stands for the PDMS concentration in hexane. The thickness of the PDMS layer was measured by ellipsometry and profilometry. Ellipsometry was used for accurate measurement for the 0.02 and 1 wt % layers, and for thickest PDMS layer (50 wt %), profilometer was used. The thicknesses were 4.5 μm (DR1g-PDMS₅₀), 65 nm (DR1g-PDMS₁), and 20 nm (DR1g-PDMS_{0.02}). The DR1g-PDMS bilayer was photo-patterned using light interference lithography in the Lloyd's mirror configuration which induces mass migration in DR1g and surface deformation of the PDMS coating to form the SRGs. The periodicity of the interference pattern was determined by the wavelength and the angle between the mirror and the laser beam. By varying the angle, SRGs with different periodicities can be achieved (roughly in the range 300 nm-10 μm). Here, the periodicity was set to 1 μm, as this periodicity has been previously used in controlling the alignment of epithelial cells for the same material. In situ monitoring of the SRG formation within the different DR1g-PDMS bilayers was conducted via diffraction efficiency (DE) measurements. The thickness of the DR1g film (480±20 nm) was chosen to be large enough so that the SRG formation was independent from small variations in the layer thickness. Thus, the differences in DE were solely due to differences in the PDMS layer. The samples were imaged with atomic force microscopy (AFM), to confirm the SR formation.

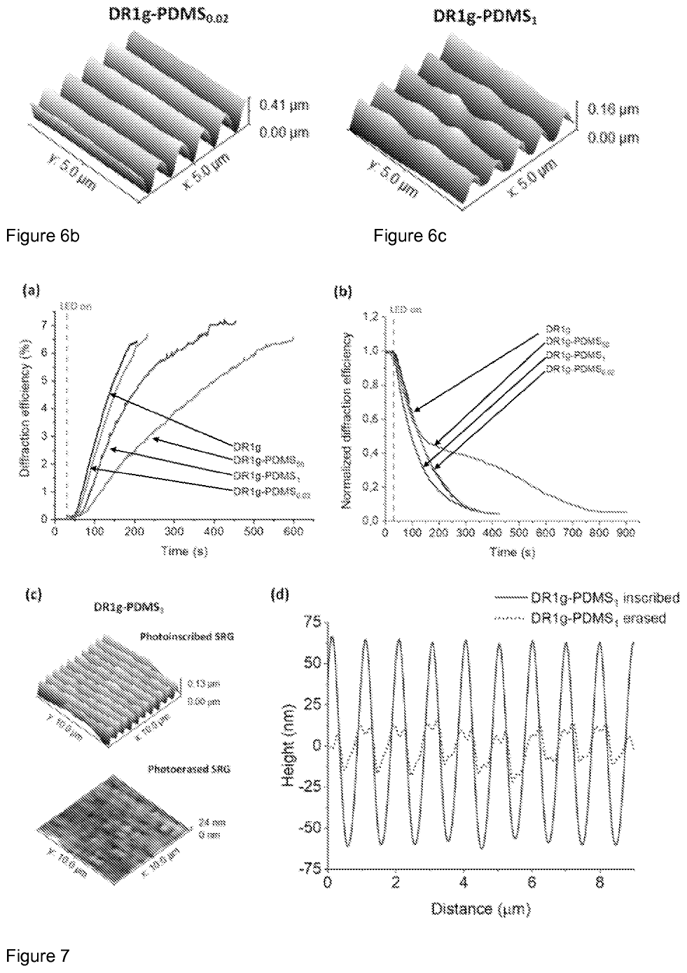

The DE curves during SRG inscription on the DR1g-PDMS bilayers are shown in FIG. 6a. From these images, it can be observed that the DE systematically decreases with increasing PDMS layer thickness. AFM imaging confirmed the formation of SRGs for DR1g-PDMS_{0.02} and DR1g-PDMS₁ with the expected periodicity of 1 μm (FIG. 6b,c). Surface modulation depth was over 400 nm for DR1g-PDMS_{0.02}, while for DR1g-PDMS₁, the modulation depth was significantly decreased and reached 160 nm. With DR1g-PDMS₅₀, SRGs were not formed onto the outer surface of the PDMS coating since no sinusoidal pattern could be observed with AFM. The 8% DE can be attributed to the grating formation at the DR1g/PDMS interface. When the azobenzene-containing film is between the glass substrate and a protective coating, it has stronger constraints to move efficiently. The SRG formation requires material mass migration, thus the presence of thick PDMS layer resulted in increased hindrance to the complex stress field DR1g is subjected to during SRG formation. Accordingly, the PDMS coating with thickness below 100 nm on top of a thin DR1g film does not inhibit the formation of SRGs but alters the dynamics of its formation.

After the SRGs are formed, the topography is stable for at least a year at temperatures below the glass-transition temperature of DR1g (71° C.) but can be erased thermally or with a uniform light beam with wavelength matching the absorption band of DR1g. As direct heating cannot be localized and is not compatible with cell culture conditions, it is preferable to erase the SRGs with visible light (e.g., 530 nm LED). To study the dynamics of the SRG erasure, the DE was monitored during the erasure for samples exhibiting the same initial DE value (ca 7%, FIG. 7a). The erasure dynamics are shown in FIG. 7b. All samples reached similar (ca 0.5%) DE value at the end of the process, hence the PDMS layer thickness does not seem to affect the effectiveness of the erasure process in terms of DE values. However, clear differences in erasure dynamics can be observed between the different PDMS thicknesses. Interestingly, the DE decreased systematically faster for DR1g-PDMS₁ compared to other samples, suggesting that the PDMS layer with proper thickness can even accelerate the topography erasure. With DR1g-PDMS₅₀ the erasure dynamics differed from other samples and the DE decreased relatively slowly, in a two-step process.

The samples were imaged with AFM, which confirmed that the topography was reduced up to 85% of the initial value for DR1g-PDMS₁ as FIG. 7c shows. The surface profiles in FIG. 7d further highlight the difference between erased and non-erased topographies. The decrease in modulation depth was 70% for DR1g-PDMS_{0.02}. Even if the DE-values reached a similar value, AFM shows that with DR1g-PDMS₁, the erased topography reached a lower modulation depth. This served as the motivation to select DR1g-PDMS₁ for cell culture studies. With deeper gratings, the erasure with uniform laser beam is less effective in comparison to lower SRGs. Thus, the complete erasure of the topography might be more difficult achieve for DR1g-PDMS_{0.02} surface, as determined from the higher remnant gratings observed on this sample. With DR1g-PDMS₁, the grating depth favored a fast erasure process, leading to a lower residual topography, limiting the irradiation time needed during cell-growth experiments. The gratings were also deep enough to induce cell response in terms of alignment. The thicker PDMS layer may acts as a better protective layer to separate the DR1g layer from the cells.

The DR1g layer was also coated with Parylene C (, i.e., poly(chloro-para-xylylene) by chemical vapor deposition polymerization (CVD). CVD of parylene C was mediated by four secondary chambers inserted in the main deposition chamber each deposition run. Such secondary chambers presented a small orifice at the top, whose function was controlling the deposition rate of the reactive monomers and thus, fine tuning the thickness of the deposited layer. In general, the thickness of the deposited parylene film is directly proportional to the mass of dimer loaded into the machine. However, for ultra-low thick films (<100 nm) a reduction of dimer mass would lead to a not well controllable and reliable deposition process (due to a very short and unstable pressure). By exploiting the effusion of parylene molecules through a hole whose size is smaller than the mean free path of parylene monomers (Knudsen number greater than 1), the thickness of the deposited layer can be reliably obtained by allowing only a precise fraction of the reactive monomer to enter the secondary chamber.

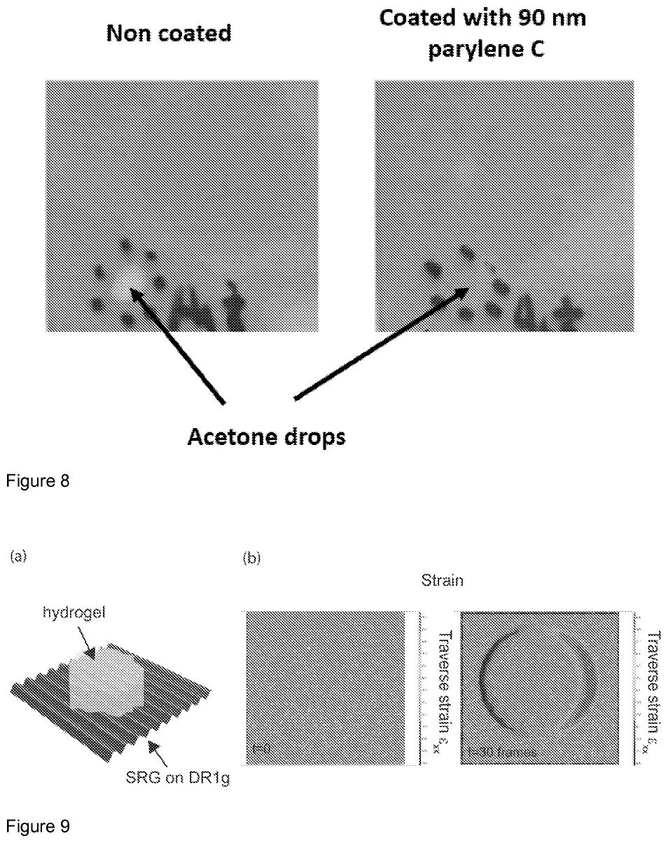

The prepared substrates were characterized by stylus profilometry and showed thicknesses in the range 13 nm-415 nm. The formation of SRG was then tested as described above for PDMS-coated samples. The samples showed the formation of SRG in all the samples with thicknesses below 90 nm. The barrier properties of the parylene C layer were also tested; a permeability test was performed by depositing a 1 μl drop of different organic solvents (acetone, ethanol, and isopropanol) and water over the samples' surfaces. In the lapse of a few seconds to three minutes the solvent drops either dissolved a portion of the DR1g layer or stayed intact on top of the samples' surface until finally evaporating. The samples above 55 nm were, as expected, resistant to water penetration but also offered the best protection against organic solvents (in particular ethanol and isopropanol). The most aggressive solvent tested was acetone and it was used to define the upper thickness limit for the parylene layer, on which SRG could be formed and that, at the same time, showed a consistent and reliable protection against acetone penetration (FIG. 8).

A polyacrylamide hydrogel layer was also used to coat DR1g substrates (FIG. 9a,b). The polyacrylamide was loaded with fluorescent microparticles emitting red light (607 nm). The thickness of the hydrogel was estimated to be 100 μm. DR1g was then photostimulated by a 488 nm laser at a laser scanning confocal microscope (LSM 780, Zeiss) in user-defined regions of interest (ROIs). In those ROIs, the photosensitive material flow provoked a corresponding deformation in the hydrogel, as shown by the fluorescent microparticles captured by time lapse microscopy and analyzed by digital image correlation (DIC). For the tested hydrogel (2.8 kPa), the strain was transmitted inside the hydrogel, at least as far as 50 μm deep. This experiment demonstrated that, in principle, the light-induced surface deformation of DR1g can be used to stimulate a soft material, such as a hydrogel, locally and mechanically. Such hydrogel could be loaded with cells or cells could be cultured of the hydrogel surface.

### SRG-Guided Cell Alignment

Cells sense physical properties of their environment and mechanical forces at their surface, but these forces are transduced also deeper into the cells, even to the nucleus. The main sites for sensing are cell-ECM contacts, mainly focal adhesions, which are multiprotein complexes at the cell membrane. The formation of focal adhesions at the cell-ECM interface regulates the cell attachment, alignment and migration. A calcium-dependent transmembrane protein, E-cadherin, is one of the molecules found at the cell-cell contact sites. E-cadherin is present especially in adherents junctions and plays a key role at the cell-cell interface during formation of tight and polarized epithelium.

To study whether the microtopography on DR1g-PDMS₁ bilayer could guide collective cell alignment, Madin-Darby canine kidney type II (MDCK II) epithelial cells were seeded on the SRG and their alignment to the underneath microtopography was studied. This cell line provides a good model for studying cellular collective behavior.

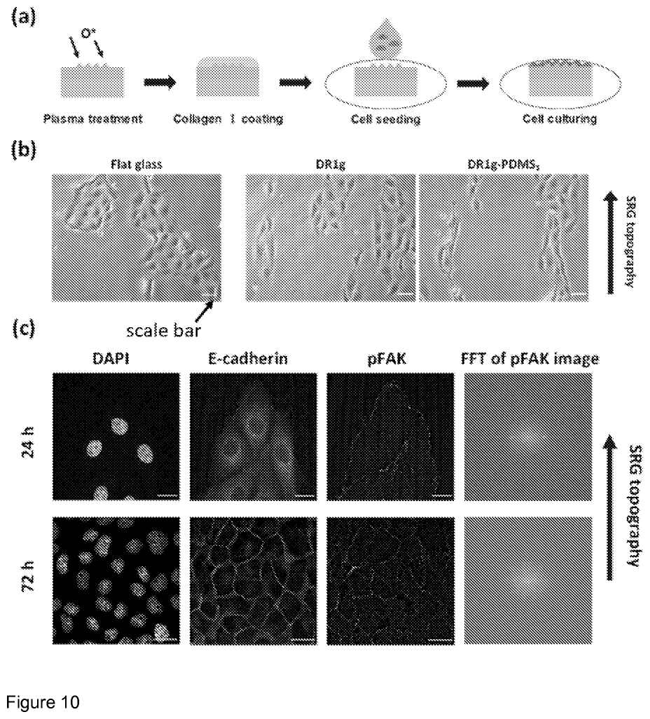

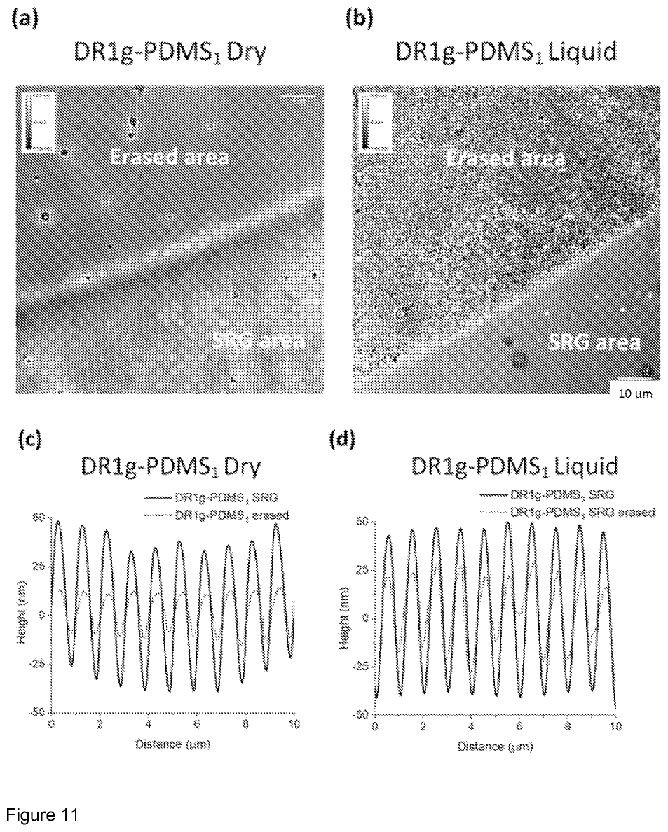

While the mechanotransduction of single cells on micro topographies has been largely investigated, such behavior has not yet been fully characterized for cell collectives, for which concerted movements happen without complete disruption of their cell-cell contacts. FIG. 10a shows the schematic representation of the sample preparation. Briefly, the DR1g and PDMS were subsequently spin-coated to form the bilayer structure and the SRGs were inscribed as previously described. After patterning, the surface was made hydrophilic with an oxygen plasma treatment to improve protein attachment to the surface. The surface was then coated with collagen I to improve cell adhesion onto the surface and MDCK II cells were seeded on the samples and cultured up to 72 h. The cell migration along the microtopography was followed by time lapse microscopy. Cells aligned along the microtopography already during the first 24 h post seeding on DR1-PDMS₁. After 24 h, the cells were forming small colonies, elongating along the pattern direction on both bare DR1g and DR1g-PDMS₁, indicating that the PDMS layer does not inhibit cells from sensing the underneath topography (FIG. 10b). The cells formed a confluent cell monolayer at 72 h time point.

The cellular response to the microtopography in terms of cell-material and cell-cell interactions was further investigated by immunolabeling the MDCK II cells at different time points. The nuclei were stained with DAPI for distinguishing single cells. The cell-cell interaction was studied by detecting intracellular localization of E-cadherin. After 24 h from cell seeding, the cell nuclei were round, but the cells had an elongated morphology along the surface microtopography as observed from E-cadherin localization (FIG. 10c). In addition, E-cadherin accumulated in the cytoplasm, thus the cells were not yet forming mature cell-cell junctions. At 72 h timepoint, the morphology of the cells was less elongated than at the 24 h timepoint. After 72 h, cells were forming a uniform cell layer and E-cadherin localized to the cell-cell interface, showing strong cell-cell interactions on the bilayer surface. The loss of E-cadherin, instead, indicates epithelial-mesenchymal transition (EMT), where epithelial cells lose their phenotypic characteristics and transform to nonpolarized mesenchymal cells, which are more motile and potentially invasive. Since E-cadherin was localized at the cell-cell interface, cells on the bilayer surface were forming a tight epithelium layer after 72 h.

Focal adhesion kinase (FAK) is one of the first molecules present in focal adhesion development and its phosphorylation indicates the formation of mature focal adhesions. Thus, morphological parameters of focal adhesions were studied by immunolabeling phosphorylated FAK (pFAK). Focal adhesions were observed at the cells edge in the basal plane after 24 h from seeding and their distribution was further analyzed by using fast Fourier transform (FFT). FFT converts the spatial image information into frequency space, where periodic features are emphasized yielding a specific pattern of frequencies. The analysis showed that first-order frequency peaks can be detected after 24 h from cell seeding (FIG. 9c, FFT of pFAK image) showing periodic distribution of the image features (pFAK). After 72 h, pFAK was still observed at the cell edges, though after forming uniform cell layer, cell movements are more restricted. In the FFT for the focal adhesion channel the first-order frequency peaks were still visible, indicating that focal adhesions were periodically distributed, and cells were still perceiving the information from the topographical cue.

Erasure of SRG Topography with Live Cells. Instead of using the LED, the microtopography was erased with a fluorescent lamp of a confocal microscope (filtered in the blue region of the visible spectrum), which enables the observation of live cells right after the measurement. This setup was deemed practical for biological environment since most microscopes can be equipped with environment control, suitable for live cell culture. The erasure was first conducted in dry and liquid environments at room temperature without cells, to set the erasure parameters. Illumination with the fluorescent lamp clearly resulted in a distinguishable circular area in both dry and aqueous environment, as seen from bright field images and digital holographic microscopy (DHM) images, which yield quantitative results about the surface profile (FIG. 11a,b). DHM was used to monitor the surface, allowing a fast and quantitative characterization of the surface topography over a bigger area compared to AFM. DHM images showed reproducible decrease in modulation depth within 5 min of irradiation. In dry conditions, the modulation depth decreased 75% from the initial value (FIG. 11c), indicated by surface roughness decrease from 56 nm to 14 nm. In liquid environment the modulation depth of the erased area decreased by 50% (FIG. 11d), and in addition, the (partially) erased surface was significantly rougher and exhibited round surface features.

MDCK II cells were seeded on the SRG topography and cultured for 24 h prior to the erasure, to allow cell orientation along the microtopography. The samples were illuminated with the fluorescent lamp of a confocal microscope with medium on top at 37° C. in a humidified atmosphere for 5 min, fixed after 2 h from erasure and immunolabeled. The partial photo erasure was confirmed by DHM after cell removal by trypsin treatment. In the presence of the PDMS layer, the erasure was more uniform in comparison to the bare DR1g, yielding significantly lower number of the round surface features described above. The possible phototoxicity on cells was also studied. For this experiment, cells were seeded on samples in which DR1g was spin-coated to the bottom side of the glass coverslip and glass substrate was at cell-material interface. Such control sample ensured that a similar light intensity reached the plane of the cells as in the erasure process, but no topographical change could be produced at the cell adhesion sites. The control samples were illuminated with the fluorescent lamp for 5 min, and after 3 h form erasure Live/Dead viability/cytotoxicity assay was performed. No major acute phototoxic effects on cell viability could be observed as no dead cells could be seen in the erased areas similarly to the non-erased areas. When studying phototoxicity effects on cell morphology, PDMS was spin-coated on the other side on top of the control sample, at the cell-material interface, to ensure similar adhesion properties. No significant difference in cell morphology could be observed within 2 h from irradiation.

The cell groups had a less spread morphology and smaller size after erasure, which might indicate partial loss of substrate attachment after the topography changes. In addition, pFAK was observed to be more concentrated in the cell center rather than in cell edges after the erasure. When microtopography was erased underneath a uniform epithelial cell layer, no significant morphology change could be observed. This observation suggests that, when strong cell-cell connections are formed, epithelial cells in a monolayer do not immediately rearrange as a response to loss of guiding surface topography, at least within 2 h post-erasure. Quantification of the focal adhesion orientation was conducted similarly as described above. The orientation data showed that focal adhesions were more randomly oriented after erasure with small cell groups. However, no differences could be observed in the case of confluent cell layer. This indicates that smaller cell groups can sense the light-induced topographical change and re-orient the focal adhesions accordingly. Erasure seemed to have no effect on elongation and area of the focal. Even if the topography erasure was only partial with the lamp of the confocal microscope in the presence of liquid, the microtopography and the roughness of the surface could be changed. The topography change affected the morphology and focal adhesion orientation with small cell groups. However, no collective morphological response or focal adhesion orientation could be observed at least during the time span of 2 h. The cells remained attached and viable on the erased surface after irradiation.

### Conclusions

The platform presented here consists of a light-responsive azobenzene containing film and a thin PDMS or parylene C coating, which allowed independent control of the light responsivity and the stability of the material in cell culture environment. Together these layers formed a bilayer structure, which allowed surface topography modification with light-induced movements of azobenzene-containing film. The SRG topography was efficiently photo-inscribed and -erased in the presence of PDMS and parylene C layer. When MDCK II epithelial cells were seeded on photopatterned systems, the SRG topography could guide focal adhesion orientation along the surface topography still after formation of uniform epithelial layer. The surface topography could be altered in the presence of live cells with a fluorescent lamp of a confocal microscope, enabling noninvasive control over the surface topography. Despite the SRG topography erasure was only partial, the topography could still be changed without causing cell detachment or cell death. Thus, light-mediated erasure is a strategy to control the material topography dynamically for real-time cell experiments, which can be conducted with conventional microscopy setups. The platform could be further patterned with proteins, enabling individual control of the topographical and biochemical cues and further functionalization for different applications.

### Experimental Section

Sample Preparation. Polymer is PDMS. A bilayer of azobenzene-containing Disperse Red 1 molecular glass (DR1g, Solaris Chem Inc.) and polydimethylsiloxane (PDMS, SYLGARD 184, Dow) was prepared on square glass coverslips by spin coating (Laurell Technologies Corporation). The glass coverslip was first ultrasonicated twice in acetone for 10 min. Solution of DR1g with concentration of 9% (w/v) in chloroform was prepared. The solution (35 μl) was deposited on the glass coverslip (22×22 mm²) at 1500 rpm for 30 s. PDMS was prepared by mixing prepolymer silicone elastomer base and curing agent in 10:1 ratio. The uncured PDMS was diluted in n-hexane to create 50, 1, and 0.02 wt % solutions. The solutions were dispensed over the thin DR1g film at 6000 rpm for 150 s and cured at 55° C. for 1.5 h. Samples for thickness measurement were first prepared by spin coating PDMS solution on a silicon substrate as described above. Thickness of the produced PDMS film was measured with reflection ellipsometry (J. A, Woollam VASE). The 50 wt-% PDMS solution formed too thick film for ellipsometry measurement, thus the thickness was measured with Stylus profilometry (Veeco Dektak 150). For both techniques the resolution limit is in sub-nanometer range.

Sample Preparation. Polymer is Parylene C.

A bilayer of azobenzene-containing Disperse Red 1 molecular glass (DR1g, Solaris Chem Inc.) and parylene C (Galentis Ltd.) was prepared on square glass coverslips by spin coating the Disperse Red 1 as shown above, followed by chemical vapor deposition (Para Tech Coating Inc.) of the parylene C, using the effusion-based method described elsewhere. The orifices that connected the inside of each secondary deposition chamber with the larger, principal machine chamber, were square holes in the range of 200 μm to 8000 μm lateral size. The final thickness of the films was estimated with a stylus profilometer (Bruker Dektak XT). For each deposition run, 2 g of dichloro-p-cyclophane dimer were loaded into the deposition system serving four cylindrical secondary chambers with internal surface area 19210 mm².

Sample preparation. Polymer is polyacrylamide hydrogel: Round glass coverslips (13 mm) were washed with 2% Hellmanex solution in a ultrasonicating bath for 30 min, washed with abundant deionized water, and carefully dried. Glass coverslip passivation was achieved with grafting PLL-PEG. A drop (10-30 μl) of 0.1 mg/ml PLL-g-PEG in PBS was deposited on the coverslips and let react for 30 min. The substrates were then washed with abundant deionized water. The reagents solution was prepared as follows: acrylamide (10 wt %), Bis-acrylamide (0.03 wt %), fluorescent microparticles (0.04 wt %), N,N,N′,N′-tetramethyl ethylenediamine (TEMED, 0.02 vol %), and ammonium persulfate (0.1 wt %) were dissolved in PBS. The gelling solution was then pipetted over a DR1-g coated glass coverslip and covered with the passivated coverslip for 15 min. The expected elastic modulus of the hydrogel is 2.8 kPa and the thickness 100 μm.

Surface Relief Grating Inscription and Erasure. The bilayer structures were photopatterned with interference lithography in Lloyd's mirror configuration. Inscription of surface relief gratings (SRGs) was done using a 488 nm continuous-wave laser (Coherent Genesis CX488-2000) with circular polarization and an intensity of 500 mWcm⁻² over an area of 0.50 cm². The microtopography period Λ was set to 1 or 1.5 μm and it was determined by Λ=λ/2 sin ϑ, where λ is the wavelength of the laser and ϑ is the angle between the mirror and the laser beam. Erasure of the SRGs was done with 530 nm LED and beam was focused directly on SRG topography with intensity of 100 mWcm⁻². The inscription and erasure of the SRGs was monitored with a low-power (1 mW) 633 nm He—Ne laser and the diffraction efficiency of the first order diffracted beam was measured.

Cell Culture. Epithelial Madin-Darby canine kidney type II (MDCK II) cells were used for this study. They were cultured at 37° C. under a humidified atmosphere with 5% CO₂ in a culture medium consisting of MEM GlutaMax (Gibco) supplemented with fetal bovine serum (10%) and penicillin/streptomycin (1%). Before cell seeding, the samples were sterilized under UV light for 40 min. The samples were coated with 50 μg ml⁻¹ monomeric rat tail type I collagen solution (Thermo Fischer Scientific) in 0.02 N acetic acid for 40 min.

Immunolabeling. Cells were fixed with 4% paraformaldehyde for 10 min, washed with PBS, permeabilized for 10 min with permeabilization buffer (0.5% BSA, 0.5% Triton-X 100 in PBS) and blocked for 1 h using 3% bovine serum albumin in PBS. The samples were labeled with rabbit anti-pFAK (1:200, Abcam, #ab81298) and rat anti-Uvomorulin/E-Cadherin (1:100, Sigma-Aldrich). Secondary antibodies used were anti-rat-Alexa 568 (1:200, Thermo Fisher Scientific #A110077) and anti-rabbit-Alexa 647 (1:200, Thermo Fisher Scientific, #A21244). Actin cytoskeleton was labeled using 488-phalloidin (1:50, Sigma-Aldrich #49 409). Samples were mounted with ProLong Diamond antifade mountant with 4′,6-diamidino-2-phenylindole (DAPI) (Thermo-Fisher Scientific, #P36935), which stains the cell nuclei.

Optical Imaging. Samples were imaged with an optical (Zeiss) and confocal microscope (Nikon A1R laser scanning confocal microscope, Nikon Instruments Europe BV). For confocal microscopy the laser lines used were 405, 488, 561, and 633 nm. For each image, the laser intensity was adjusted to avoid photobleaching, and detector sensitivity was tuned to optimize the image brightness. A 60×/1.4 Plan-Apochromat oil immersion objective and 20×/0.8 Plan-Apochromat air immersion objectives were used to capture 1024×1024 pixel images. The data was in the form of 3D z-stacks, which included 30-40 slices each with 150-250 nm interval. Time lapse microscopy was performed with EVOS FL auto (Thermo Fisher Scientific).

Topography Erasure with Confocal Microscope. The SRG topography was erased with a LSM780 laser scanning confocal microscope (Zeiss). Plan-Apochromat 20/1.4 water immersion objective was used during erasure. The samples were either in dry, liquid or cell culture environment during the irradiation. The samples were illuminated for 5 min with a fluorescent lamp filtered in the blue region (470±40 nm) with intensity of 1.5 Wcm⁻². Bright field images of the topography were captured before and after erasure. With MDCK II cells, the samples were irradiated with the fluorescent lamp, after which the cells were detached from the sample with trypsin for surface characterization or fixed after 2 h for immunolabeling.

Live/Dead Viability Assay. MDCK II cells were seeded on photopatterned bilayer and cultured on top of the samples for 24 h. Topography was erased as described above. After 3 h from the erasure, cells were washed with PBS and stained using LIVE/DEAD Viability/Cytotoxicity Kit *for mammalian cells* (Thermo Fischer Scientific) by adding 600 μl LIVE/DEAD reagent solution on each sample, containing 0.50 μl/ml calcein AM and 2 μl/ml ethidium monodimer−1 in PBS. The samples were incubated at 37° C. under a humidified atmosphere with 5% CO₂ for 30 min. Following incubation, reagent solution was aspired and 600 μl PBS was added to prevent the cells from drying. The samples were imaged with a confocal microscope (Nikon A1R laser scanning confocal microscope) using 488 nm and 561 nm laser lines. A 20×/0.8 Plan-Apochromat air immersion objective was used to capture 1024×1024 pixel images.

Image and Statistical Analyses. The distribution of focal adhesions was analyzed with fast Fourier transform of the focal adhesion channel using FFT plugin in ImageJ. Prior to generating FFT image, circular region of 900 pixels was cropped and the FFT image was generated from this region. ImageJ was used to measure the elongation, area and orientation of focal adhesions. Further analysis of focal adhesion elongation and orientation was done with MomentMacroJ v1.4B script (https://www. hopkinsmedicine.org/fae/mmacro.html). The graphs in FIG. 3d-f represent average of 100 quantified focal adhesions from 10 separate images. In FIG. 5c,d and FIG. 7c the graphs represent average of 30 quantified focal adhesions from 2 separate images. Prior to analysis, the focal adhesion images were processed to remove pixel noise. The principal moments of inertia were measured (i.e., maximum and minimum) and the cell elongation was defined as the ratio of these values (maximum/minimum). Higher values indicate more elongated focal adhesions. The orientation of the focal adhesion was defined as the angle between the surface pattern direction and the maximum axis. Statistical analyses were done with Origin, version 2019b (OriginLab Corporation) and MATLAB. We estimated the statistical power of the test for experiments which had less than 100 hundred focal adhesion quantified. We estimated that the statistical differences were significant with actual power values over 75%. Our data was found to have a nonnormal distribution, thus nonparametric Kruskal-Wallis test with Bonferroni and Dunn-Sidak post hoc tests were used to evaluate the statistical significance.

The specific examples provided in the description given above should not be construed as limiting the scope and/or the applicability of the appended claims.

1-30. (canceled)

31. A patterned system for cell culturing obtainable by subjecting one or more areas of an azobenzene-containing layer of a system comprising a support structure, an azobenzene-containing middle layer and a top layer comprising a protective polymer selected from elastomer, hydrogel and parylene, preferably parylene C to light generated by a laser, preferably a continuous wave laser, wherein wavelength of the light is 430 nm-530 nm and intensity of the light is preferably 1 W cm²-5 W cm² thereby producing topographical features to the system.

32. The patterned system according to claim 31 wherein thickness of the azobenzene-containing layer is from 50 nm to 5 μm and thickness of the top layer is from 20 nm to 100 μm, preferably from 20 nm to 200 nm.

33. The patterned system according to claim 31 wherein thickness of the azobenzene-containing layer is from 50 nm to 5 μm and thickness of the top layer is from 50 nm to 100 μm.

34. The patterned system according to claim 31 wherein the support structure is selected from cell culture disc such as petri dish, microscope coverslip and a well plate.

35. The patterned system according to claim 31 wherein the elastomer is siloxane.

36. The patterned system according to claim 35 wherein the siloxane is PDMS.

37. The patterned system according to claim 31 wherein azobenzene is N-ethyl-N-(2-hydroxyethyl)-4-(4-nitrophenylazo)aniline.

38. A method for reversibly inscribing topographies on an azobenzene-containing material of a system comprising a support structure, an azobenzene-containing middle layer and a top layer comprising a protective polymer selected from elastomer, hydrogel and parylene, preferably parylene C the method comprising scanning laser beam over the top layer wherein wavelength of the light is 400 nm-600 nm, preferably 430 nm-530 nm and intensity of the light is 1 W cm⁻²-5 W cm⁻² or projecting an interference pattern of laser light to the material wherein wavelength of the two interfering laser beams is 400 nm-600 nm, preferably 430 nm-530 nm, and intensity of the two interfering laser beams is 100 mW cm⁻²-600 mW cm⁻².

39. A method for erasing the topographical features of the patterned system according to claim 31, the method comprising subjecting the topographic features to 400 nm-600 nm, light, preferably to 460 nm-530 nm light generated by a laser, preferably a continuous wave laser, or generated by a fluorescent lamp or generated by LED.

40. The method according to claim 39 wherein intensity of the light generated by the laser, fluorescent lamp or the LED is from 1 W cm⁻² to 5 W cm⁻².

41. Use of the patterned system according to claim 31 as a cell culture platform.

42. A method for cell culturing, wherein the cells are cultured on the patterned system according to claim 31.

43. The method according to claim 42, comprising the following steps
a) providing the patterned system,
b) coating top layer of the patterned system with cell adhesive proteins, and
c) seeding cells onto the cell adhesive proteins.

44. The method according to claim 42 wherein the cells are selected from epithelial, fibroblasts, endothelial, neurons, mesenchymal stem cells, astrocytes, cardiomyocytes, and cancer cells.

45. The method according to claim 43 wherein the cell adhesive proteins are selected from collagen, fibronectin, and laminin.

46. The method according to claim 42 comprising oxygen plasma treating prior to step b).
